(19) United States
(12) Patent Application Publication
(10) Pub. No.: US 2023/0268038 A1
(43) Pub. Date: Aug. 24, 2023

(54) PROXIMITY-BASED FILE SHARING SYSTEM AND METHOD

(71) Applicant: Medyear, Inc., (formerly Known As: Personiform Inc., dba Medyear), New York, NY (US)

(72) Inventors: Panha Chheng, San Leandro, CA (US); Tebogo Johannes Ngobeni, Pretoria Central, Pretoria (ZA); Jaikumar Verma, Dubai (AE)

(21) Appl. No.: 18/005,207

(22) PCT Filed: Jul. 13, 2021
(86) PCT No.: PCT/US2021/041372

§ 371 Date: Jan. 12, 2023

Related U.S. Application Data
(60) Provisional application No. 63/051,082, filed on Jul. 13, 2020.

(51) Int. Cl. G16H 10/60; G16H 50/80; G16H 50/30

(52) U.S. Cl. CPC G16H 10/60; G16H 50/80; G16H 50/30

(57) ABSTRACT

A computer-based method for sharing a digital file based on proximity and, in particular, for sharing a digital medical record file such as the result(s) from a virus test or a vaccine record. The method includes: (i) periodically transmitting, by a first mobile device of a first user, a geographic location of the first mobile device; (ii) periodically transmitting, by a second mobile device or a second user, a geographic location of the second mobile device; (iii) determining, when the second mobile device is within the predetermined zone of the first mobile device based on the transmitted geographic locations of the first mobile device and the second mobile device; and (iv) transmitting, when the second mobile device is within the predetermined zone of the first mobile device, a file associated with the first user to the second mobile device for use by the second user.

### CROSS-REFERENCE TO RELATED APPLICATION

This application is a National Stage application of International Patent Application No. PCT/US2021/041372, filed on Jul. 13, 2021, and claims priority to Provisional Application Ser. No. 63/051,082, filed Jul. 13, 2020, which are hereby incorporated by reference in their entirety to the fullest extent permissible by applicable law.

### BACKGROUND

Various public health conditions and situations, such as the COVID-19 coronavirus pandemic, require people to maintain a certain safe distance (e.g., 6 feet apart) from each other to avoid transmitting the disease. However, most people do not know the health condition of people around them, especially in a crowded public environment where most of the other people are strangers. It is also useful for people who are gatekeepers to events or facilities to know the health status of individuals before they enter or before they interact with them.

Thus, it would be desirable to have a system and method that enables an individual to know the health status or condition of other nearby people before the individual is at risk of exposure.

### BRIEF DESCRIPTION OF THE DRAWINGS

FIG. 1 shows an SMS sign-up (or registration or log-in) flow diagram, according to embodiments of the present disclosure.

FIG. 2A shows flow diagrams of ways to capture COVID-19 lab results or vaccine records for use in a broadcast, according to embodiments of the present disclosure.

FIG. 2B shows a screen illustration of ways to capture COVID-19 lab results or vaccine records for use in a broadcast, according to embodiments of the present disclosure.

FIG. 3 shows a diagram of a broadcast zone of a mobile device, according to embodiments of the present disclosure.

FIG. 4A shows a diagram of broadcast functionality of a mobile device, according to embodiments of the present disclosure.

FIG. 4B shows a diagram of broadcast functionality of a mobile device, according to embodiments of the present disclosure.

FIG. 5 shows an explanation and diagram of Geohash for use in a proximity-based filed sharing system or method, according to embodiments of the present disclosure.

FIG. 6A shows a data flow diagram of broadcasting a COVID-19 status or vaccine records, according to embodiments of the present disclosure.

FIG. 6B is an illustration of a “people near me” display which shows the health status of people within a predetermined broadcast region, according to embodiments of the present disclosure.

FIG. 7A shows a sample medical record file of a user indicating a low risk, according to embodiments of the present disclosure.

FIG. 7B shows a sample medical record file of a user indicating a medium risk, according to embodiments of the present disclosure.

FIG. 7C shows a sample medical record file of a user indicating a high risk, according to embodiments of the present disclosure.

FIG. 8A shows a diagram of the significance of IgM and IgG antibodies in connection with the COVID-19 virus and the sharing of medical records, according to embodiments of the present disclosure.

FIG. 8B shows a test result reference table showing nucleic acid (PCR) virus test results, IgM test results, and IgG test results in connection with the COVID-19 virus and corresponding reference condition, according to embodiments of the present disclosure.

FIG. 9A is a top-level block diagram of components of a proximity-based file sharing system, in accordance with embodiments of the present disclosure.

FIG. 9B is a block diagram of various components of the system of FIG. 9A, connected via a network, in accordance with embodiments of the present disclosure.

FIG. 10 shows a sample QR history entry with data and record file, in accordance with embodiments of the present disclosure.

FIG. 11A shows an exemplary screenshot of a mobile application of a proximity-based file sharing system, in accordance with embodiments of the present disclosure.

FIG. 11B shows another exemplary screenshot of the mobile application of a proximity-based file sharing system, in accordance with embodiments of the present disclosure.

FIG. 11C shows another exemplary screenshot of the mobile application of a proximity-based file sharing system, in accordance with embodiments of the present disclosure.

FIG. 11D shows another exemplary screenshot of the mobile application of a proximity-based file sharing system, in accordance with embodiments of the present disclosure.

FIG. 11E shows another exemplary screenshot of the mobile application of a proximity-based file sharing system, in accordance with embodiments of the present disclosure.

FIG. 11F shows another exemplary screenshot of the mobile application of a proximity-based file sharing system, in accordance with embodiments of the present disclosure.

FIG. 12A shows an exemplary screenshot of an application running on a professional user device, in accordance with embodiments of the present disclosure.

FIG. 12B shows an exemplary screenshot of a mobile application running on a user device, in accordance with embodiments of the present disclosure.

FIG. 12C shows another exemplary screenshot of a mobile application running on a user device, in accordance with embodiments of the present disclosure.

### DETAILED DESCRIPTION

As described further herein, the present disclosure provides novel proximity-based file sharing systems and methods. The file sharing systems and methods may be used to exchange files for various applications. In some embodiments, the proximity-based file sharing system and method is configured to share one or more medical records of a person using a mobile device with another person or device. Advantageously, the person may digitally share the medical record(s), including but not limited to COVID test, lab results, antibody test, vaccine records, or any other medical information, from a safe enough distance from the other person or device in order to adhere to appropriate social distancing requirements or a desired distance.

Thus, the present disclosure allows the user (i.e. a patient user or end user) to download antibody or virus test results or vaccine records from over 10,000 clinics or heath facilities/centers or self-report. Provides a “broadcast” feature (which can be automatically turned off after a preset time) that allows the user to anonymously share its COVID-19 status or vaccine records to, in some embodiments, an approximately 15 m×15 m square area around the user. Allows the patient user to see the COVID-19 statuses or vaccine records of other App users within the users broadcast range (e.g., approx. 15 m×15 m). It also allows for dynamic real-time adjustment of the user's broadcast square/range based on local rules or conditions of the virus.

In some embodiments, the present disclosure provides a risk score or COVID-19 risk score indicative of the level of risk a given person has regarding COVID-19. In some embodiments, the risk score may dynamically update based on the patient user's latest test results information, time since last test, and local, national, and global guidelines or rules, and/or other factors, information or data received, retrieved or measured.

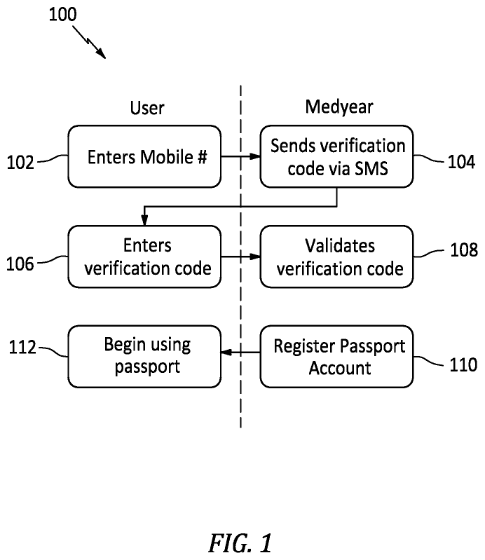

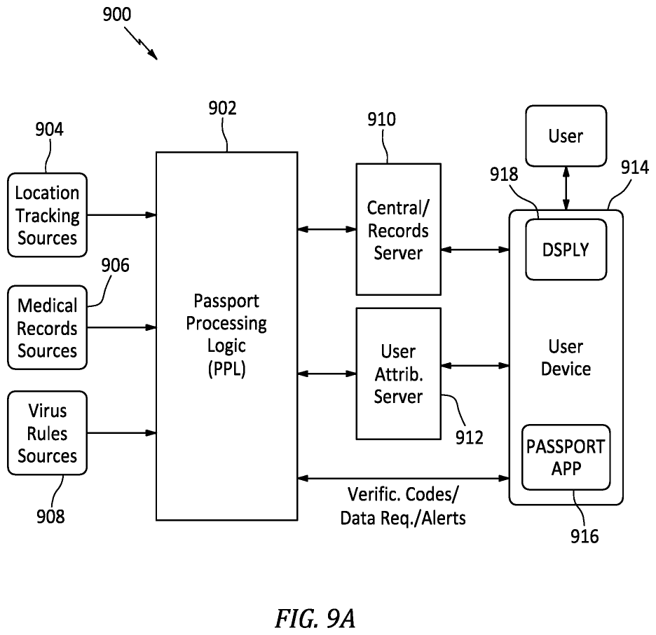

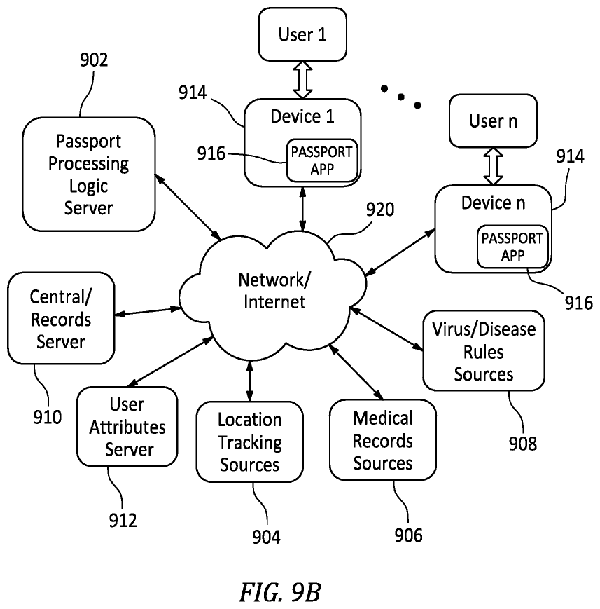

Referring to FIG. 1, an exemplary SMS sign up flow diagram process (or logic) 100 for a proximity-based file sharing system is shown according to embodiments of the present disclosure. The process 100 begins at block 102 with a patient user entering a user mobile phone number at a sign-up portal, e.g., a software mobile application (or App or Passport App) or website, which may be hosted online and accessed by the mobile device or at a different application terminal. Next, at block 104, a file sharing administrator, in this embodiment called “Medyear” or Passport Processing Logic (FIGS. 9A and 9B), which may use a central/records server, receives (or retrieves) the user mobile number and sends a verification code via SMS (or other communication protocol) to the user mobile phone number entered by the user. Next, at block 106, the user enters the verification code by the user's mobile device into the software application (Passport App) on the device (or different application terminal). Then, at block 108, the Passport Processing Logic and/or Central/Records Server, or Medyear Server, of the administrator (Medyear) validates the verification code received from the mobile device or the App. Then, at block 110, the Passport Processing Logic of the administrator (Medyear) registers a new passport account (or a new log-in or user session) and generates a unique random Person ID for this user session for the software application. Then, at block 112, the user may begin using the file-sharing application, in this embodiment referred to as “Passport” or the Passport App, where the user is permitted to access and share specific medical data through the Passport App.

In some embodiments, a unique random person identification (ID or Person ID) is generated once the user registers for the account, and every time the user logs out and registers (or logs-in or signs-in or signs-up) again, a different unique random person ID will be generated by the Passport App or the Passport Processing Logic. A user registering for the file sharing application (Passport App) by SMS allows for private registration that does not reveal the user's identity. Once registered for the file sharing application, the user may capture (or obtain or retrieve or receive) laboratory (or lab or test) results or other medical records in one or more ways.

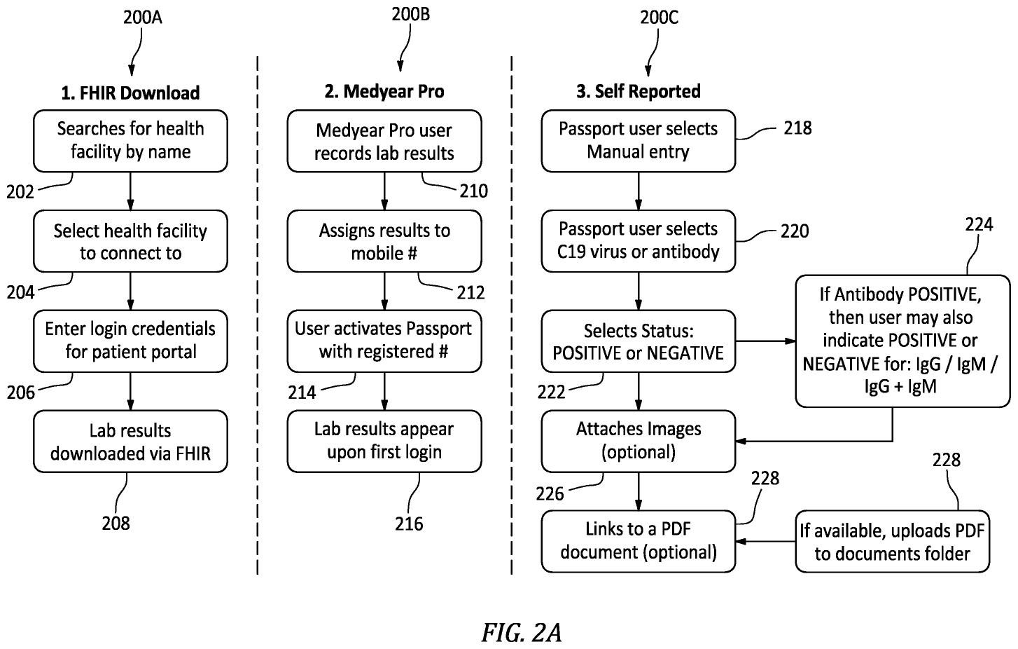

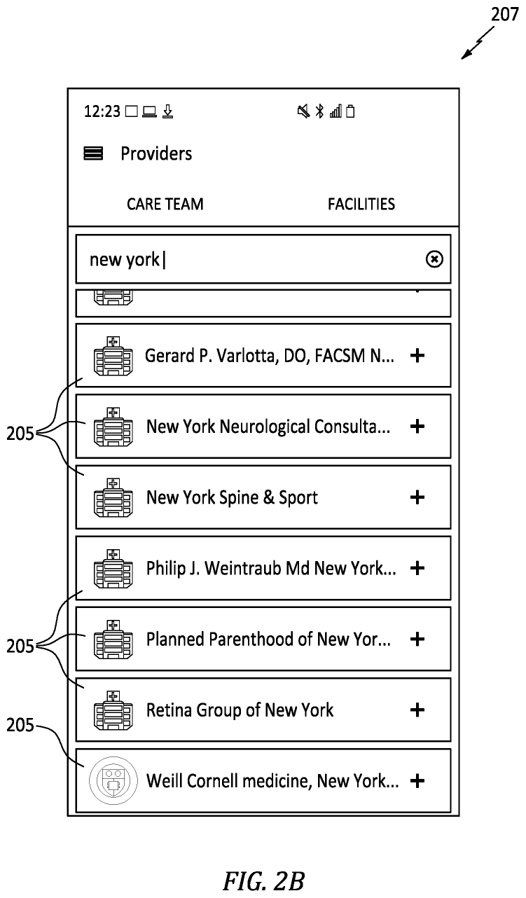

Referring to FIG. 2A, exemplary flow diagrams are shown for capturing lab or test results or vaccine records. A first method 200A is by FHIR Download, which begins at block 202 by the user searching for a health facility by name through the file sharing application on their mobile device (or other terminal). Then, at block 204, the user selects which health facility (or testing clinic) 205 to initiate a connection to, as illustrated in the screen shot 207 of FIG. 2B. Then, at block 206, the user will be prompted to enter login credentials and/or identity validating credentials for the selected health facility through a patient portal (or app) for that facility. After validation of the user's login credentials and/or identity validating credentials, at block 208, one or more lab results (in the form of a digital medical record) or vaccine records for the user will be downloaded to the Passport App via FHIR (Fast Healthcare Interoperability Resource). Fast Healthcare Interoperability Resources (FHIR) is a known standard describing data formats and elements and an application programming interface for exchanging electronic health records. The standard, as is known, was created by the Health Level Seven International health-care standards organization. In some embodiments, the digital record may be stored in the Central/Records Server and accessed by the Passport App.

A second method 200B for capturing lab results or vaccine records is by subscription to a service by a service provider or by the administrator. In this embodiment, the subscription provider may be “Medyear Pro.” First, at block 210, a Medyear Pro user (i.e. a professional user) will record lab results. Next, at block 212, the lab test results are assigned to the user mobile phone number of the user. At block 214, the user activates the application (Passport App) with the registered mobile number. Then, at block 216, the lab results will appear (auto-populate) in the Passport App application upon login. In some embodiments, the digital record may be stored in the Central/Records Server and accessed by the Passport App.

A third method 200C for capturing lab results is “self-reported” test results entered by the user. First, at block 218, an application (Passport App) user will select manual entry for “self-reporting” lab results. Next, at block 220, the user selects the type of test results to be entered by the user or self-reported, e.g., COVID-19 virus (PCR) test or antibody test (or other virus, bacteria, or pathogen test results) or vaccine records. Then, at block 222, the user selects “positive” or “negative” for the selected virus, bacteria, or pathogen test results. If antibody test result is positive, then at block 224, the user may further select whether the test results are positive for a particular antibody, for example, if for COVID-19, the user may select positive for “IgG,” “IgM,” or “IgG and IgM.” Next, at block 226, the user may optionally attach images for substantiating (or providing actual proof or evidence of) the self-reported lab or test results. Then, at blocks 228, the application links the user's selection to an optional uploaded document (e.g., a PDF document) if the user uploaded a document for substantiating the self-reported lab results. In some embodiments, the uploaded digital record/document may be stored in the Central/Records Server and accessed by the Passport App.

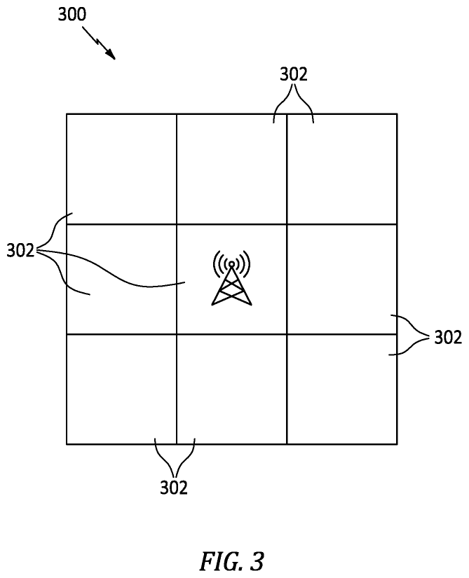

Referring to FIG. 3, a diagram of an exemplary “broadcast zone” 300 of a mobile device is shown, according to embodiments of the present disclosure. The broadcast zone 300 of the mobile device is a geographic grid of cells 302 arranged in a three-by-three (3×3) cells (or boxes or squares) arrangement with the user's mobile device located at the center cell. The cells 302 are shown as squares, but the cells 302 may be shapes other than squares and, in other embodiments, the cells 302 are not square. In some embodiments, the broadcast zone 300 is not a grid, but instead a different geographic region, e.g., a circular radius with the user mobile device located at the center of the circle. The broadcast zone 300 is determined by finding the user's location in real-time using known GPS tracking, and using known “Level-9” geo-hashing in order to determine the 3×3 cell 302 arrangement around the user's mobile device location in order to generate a predetermined broadcast zone area that is 14.34 meters by 14.34 meters.

In some embodiments, the geo-hashing level may be different, if desired, and, thus, the predetermined broadcast zone 300 area may be larger or smaller than 14.34 meters×14.34 meters. In that case, the size of the cells 302 may be other than 4.78 m×4.78 m, and may be changed, manually by adjusting the user default settings, or automatically or dynamically in real-time based on current distancing rules associated with a given virus or condition, which may be sampled by the App and updated on a real-time basis. In that case a different type of geo-hashing may be used, or the cells 302 may be grouped together to achieve the desired cell 302 size.

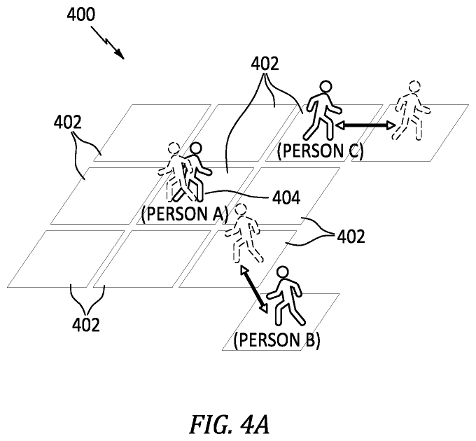

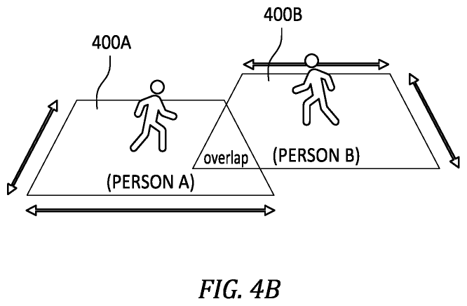

Referring to FIG. 4A, a diagram of broadcast functionality of a mobile device is shown according to embodiments of the present disclosure. The predetermined broadcast zone 400 having a three-by-three (3×3) cells 402 arrangement is shown with Person A having a mobile device located at the center 404 of the broadcast zone. Person B is shown outside of the broadcast zone 400 moving into the broadcast zone 400 and Person C is shown inside the broadcast zone 400 moving outside of the predetermined broadcast zone 400. Persons B and C each have a mobile device running the application (Passport App).

The broadcast zone 400 is shown on a two-dimensional planar surface. Person A's predetermined broadcast zone 400 includes an area that is 14.34 meters wide by 14.34 meters long directly surrounding the user's location. As discussed above, the cells 402 are not always square.

The application running on each mobile device of each Person A, B, C periodically transmits its geographic position determined by GPS technology of each respective mobile device and transmits the geographic position of the mobile device to the central server of the administrator (or other server). When a person running the application in a file receiving mode is within the broadcast zone 400 of Person A's mobile device, then Person A's medical record(s) is automatically and dynamically shared with those person(s) within the broadcast zone 400 having a device running the Passport App in a file receiving mode. The sharing is accomplished through known file transfer technology and techniques from the administrator's central server (Medyear Server, or other server). The central server (or other server) is configured to automatically share Person A's medical record(s) with the persons having the Passport App running in file receiving mode dynamically based on the updated geographic coordinates of both broadcast Person A and the receiving person(s). When Person B enters the broadcast zone 400 (i.e., the same cell area of Person A), then Persons A and B will be linked for sharing health information (or other information). When Person C exits Person A's broadcast zone 400, then Persons A and C will no longer be linked for sharing health information (or other information).

In some embodiments, the sharing may be done anonymously. For example, each user may have a user identification number that is shared with the health information (or other information) with other users. The person receiving the shared information can call out the identification number of the user(s) with acceptable test results or other shared information to proceed. In some embodiments, an avatar image or profile image of the user(s) may be shared with the other users along with the shared information. In such instances, the users receiving the shared information could correlate the users in proximity with the avatar image or profile image for allowing the user(s) to proceed if they have acceptable test results or other shared information.

Referring to FIG. 4B, another diagram of broadcast functionality of a mobile device is shown, where Person A and Person B is each shown with their respective broadcast regions or zones (e.g., about 15 m×15 m) 400A, 400B, and medical files are shared or sharable when there is overlap between the two broadcast zones.

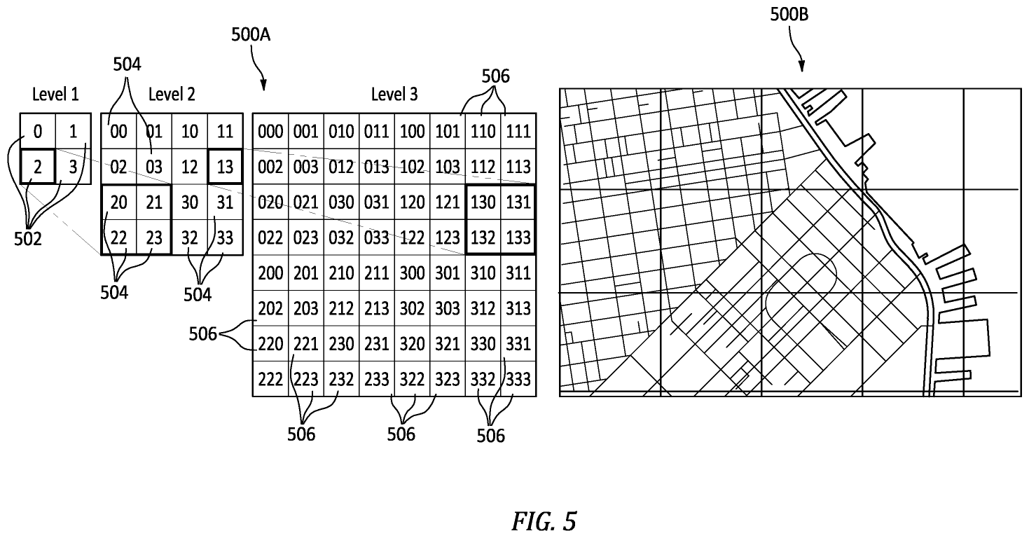

Referring to FIG. 5, a diagram 500A of Geohash is shown for use in a proximity-based filed sharing systems or methods, according to embodiments of the present disclosure. Geohash is a known public domain geocode system which encodes a geographic location into a short string of letters and digits. It is a hierarchical spatial data structure which subdivides space into buckets of grid-shaped cells 502, 504, 506, which is one of the many applications of what is known as a Z-order curve and generally space-filling curves. The cells 502 at a first level (level 1) each comprise a plurality of cells 504 subdividing the cell 502 at second level (level 2), which also each comprise a plurality of cells 506 subdividing the cell 504 at a third level (level 3). The diagram 500B shows a “level 9” Geohash, which is a precision factor of nine, generating a cell having dimensions of 4.78 m high and 4.78 m wide (and cells are not always square). The geographic depictions within each cell of diagram 500B is for illustrative purposes only and is not to scale for level 9 Geohash.

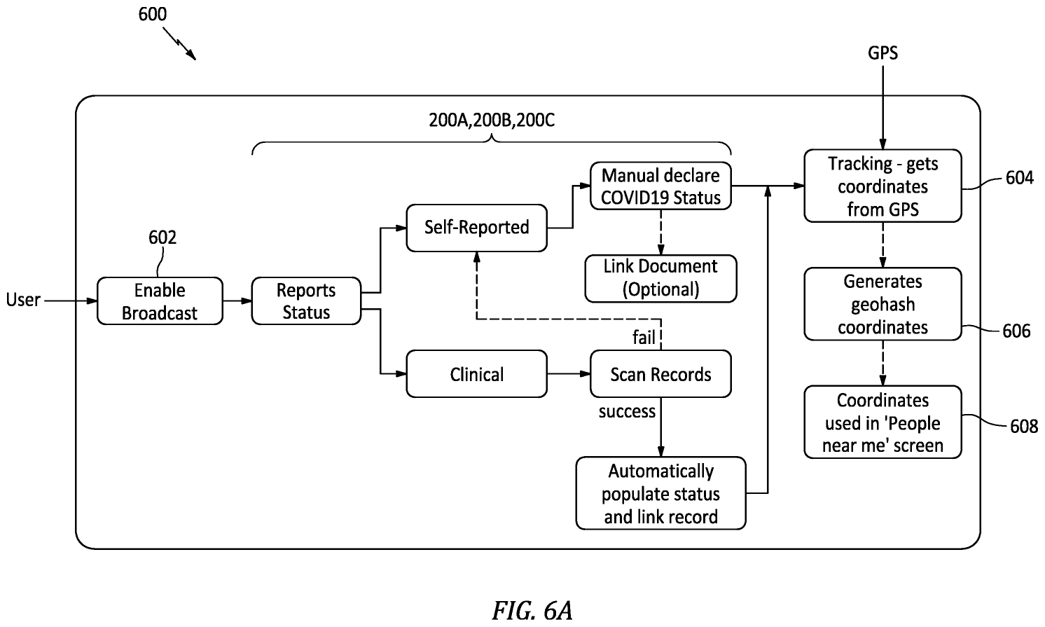

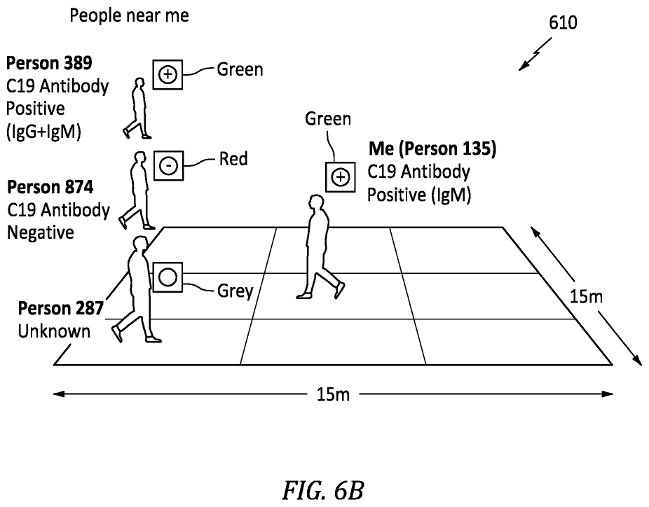

Referring to FIG. 6A, a flow diagram 600 of broadcasting a COVID-19 status is shown according to embodiments of the present disclosure. At block 602, the user enables the broadcast function of the application (Passport App) running on the user's mobile device. The application proceeds to capture lab results in one or more methods 200A, 200B, 200C as discussed above in connection with FIGS. 2A and 2B. If the application proceeds to obtain test results from a health facility (i.e., “clinical”), the application will automatically scan the records from the health facility upon entry of valid login credential as discussed above, and will automatically populate the status and link the desired records. If the application fails and is unable to retrieve records from the health facility due to invalid login credentials or other error, the application proceeds to obtain results through “self-reporting” path as discussed above. Then, at block 604, whether through “clinical” retrieval of records or through “self-reporting”, the application proceeds to perform tracking of the GPS coordinates of the user's mobile device through GPS information provided to the application from the mobile device. At block 606, the application (Passport App) will generate Geohash coordinates based on the GPS information provided. At block 608, the coordinates are used in a “People near me” screen 610 configured to be generated in the application, as shown in FIG. 6B.

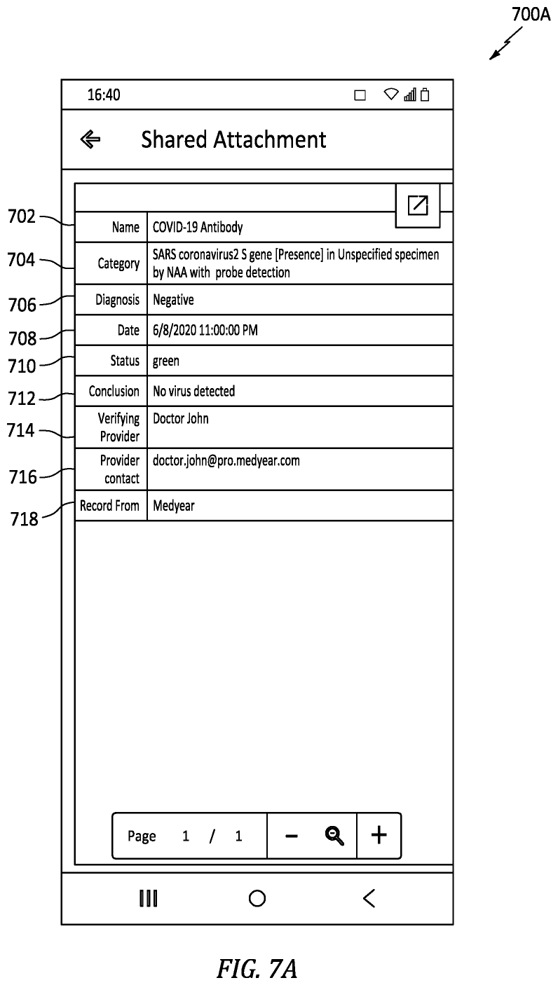

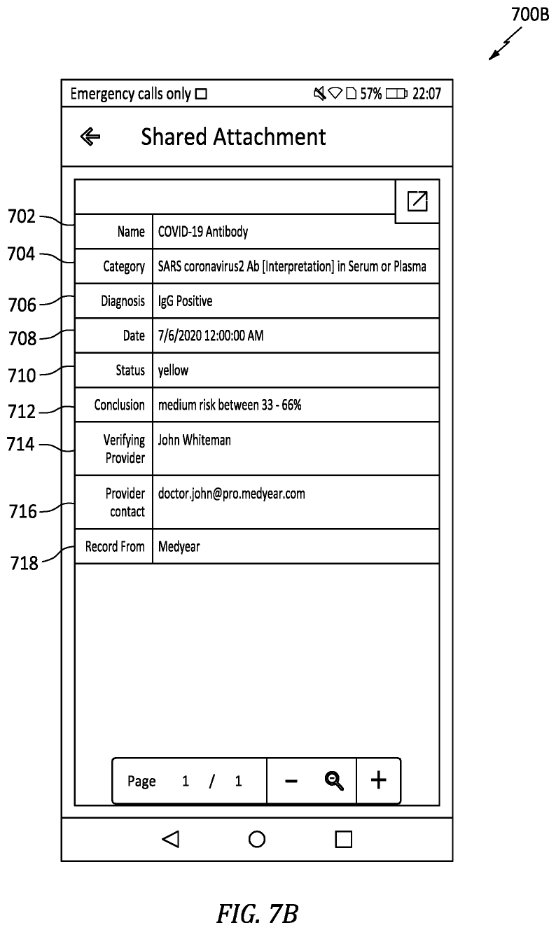

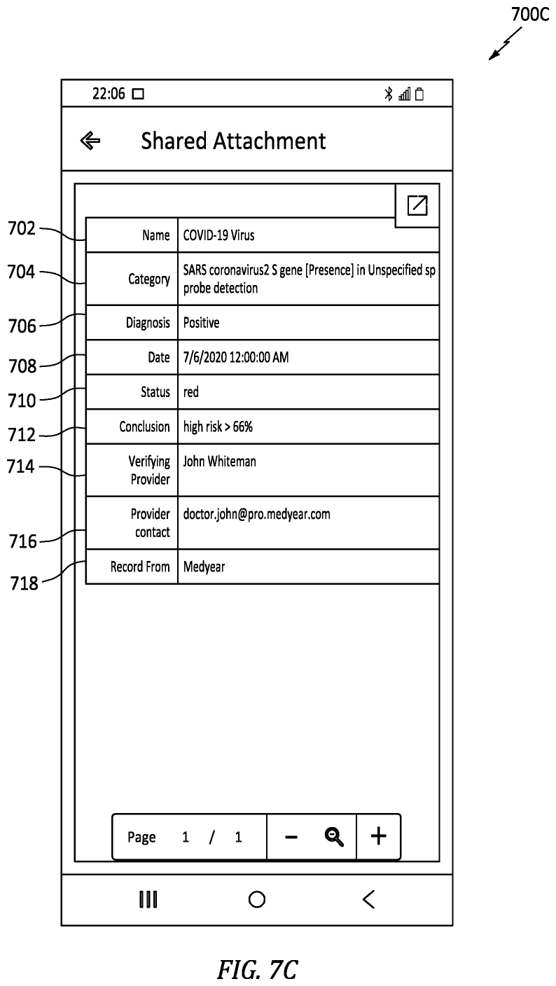

Referring to FIGS. 7A-7C, digital (or electronic) medical records or files 700A, 700B, 700C of users are shown according to embodiments of the present disclosure. The medical record files in these embodiments are medical test results record that contains a name field 702 for the test (e.g. COVID-19 Virus), a category field 704 (e.g. SARS coronavirus . . . ), a diagnosis field 706 (e.g. negative, positive), a date field 708 (e.g. 6/8/2020 11.00.00 PM), a status field 710 (e.g. green, yellow, red), a conclusion field 712 (e.g. No virus detected), a verifying provider field 714 (e.g. Doctor John), a provider contact field 716 (e.g. doctorjohn@pro.medyear.com), and a record from field 718 (e.g. Medyear). Other fields may be provided if desired. The application running on a device receiving a shared file 700A, 700B, 700C may generate a color and/or symbol based on the status field 710. For example, in this embodiment, with a status field 710 being green, the application may generate a green color and/or a green check mark on the display of the device, indicating to the user of the device receiving the shared file 700A, 700B, 700C that the user of the device sharing the medical test record 700A, 700B, 700C is cleared for entry or other privilege, or generate a red color and/or a red “x” on the display of the device, indicating to the user of the device receiving the shared file 700A, 700B, 700C that the user of the device sharing the medical test record 700A, 700B, 700C is not cleared for entry or other privilege. In some embodiments, the status field 710 may be a score (e.g. a COVID-19 score as is discussed below) or the status and/or conclusion may be determined based on a score, or there may be separate fields for status and score.

Also, in some embodiments, the test results may appear in the App in the form of a 1D or 2D bar code or QR code (or any other machine readable code or graphic), which may be scanned, e.g., by a gatekeeper or enter/exit monitor, to obtain (or to link to) a user's actual medical records or test results. The code or graphic may be scanned at a reading device that is at an appropriate social distance from the gatekeeper, e.g. a wall mounted scanner arranged six feet from the gatekeeper. In some embodiments, the gatekeeper is a person monitoring results retrieved/shared after each person's scan at the scanner. In some embodiments, the gatekeeper is a device or mechanism that is configured to allow access to each person, e.g. by opening a door or unlocking a door, if the person has medical records retrieved/shared that satisfy a predetermined threshold, e.g. a negative virus test result within the previous seventy-two hours. Also, in some embodiments, the bar code may be digitally shared and read remotely by a bar code reading device, to further protect anonymity. Also, in some embodiments, the bar code itself may turn a color based on the test result status or overall user status, which may be observed from a safe distance, such as, red, green, yellow, blue, each color indicating a particular consequence/entry result (e.g., red=no entry, green=allow entry, yellow=entry only with a mask, blue=entry only with mask and gloves). Also, in some embodiments, the test results screen (text or background) may turn a color based on the test result status.

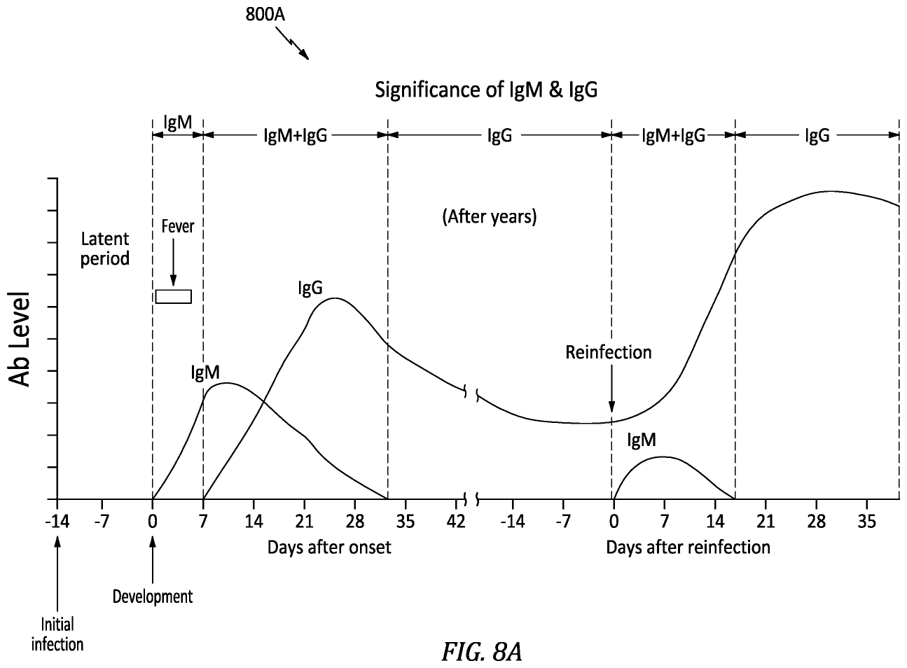

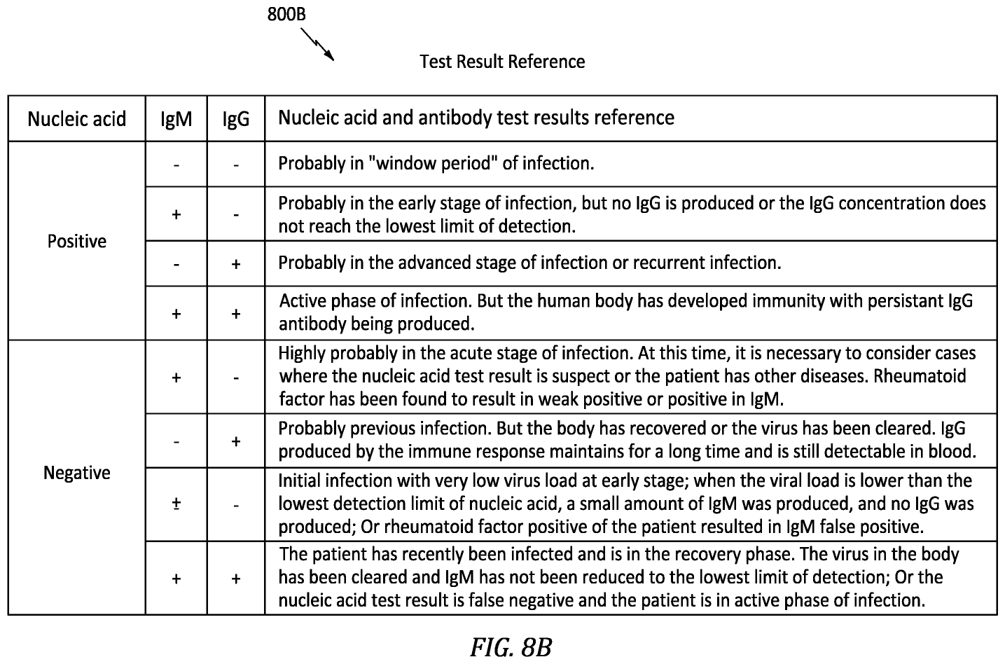

Referring to FIGS. 8A and 8B, a diagram 800A and table, respectively, of the significance of IgM and IgG antibodies (FIGS. 8A, 8B) and COVID virus test (FIG. 8B), in connection with the COVID-19 virus and the sharing of medical records is shown according to embodiments of the present disclosure. The antibody type (IgM, IgG) and level (Ab level) are important in terms of the development of the condition in the person. Therefore, knowing when a person received test results indicating whether antibody IgM and/or IgG are detected, and at what level(s), may play an important role for persons desiring to know such information, e.g., employers, venue operators, security, prisons, schools, airlines, etc. Also, knowing when a Covid virus test (PCR) was performed is useful for determining if the test results are still useful (e.g., have gone stale after 72 hrs). The test results of a user and when the test was performed is valuable information for many applications when persons are in relatively close proximity to each other. The methods and systems of the present disclosure being able to quickly and effectively share medical records with others based on proximity to others is novel and useful for many different applications.

In some embodiments, the application running on the device(s) is configured to generate a score (or COVID-19 score or a risk score, or a COVID-19 risk score) based on test results containing information about the user and other factors. For example, the application may be configured to generate a COVID-19 score based on when the user had a COVID-19 test performed, how many times the user had COVID-19 tests performed, the results of the COVID-19 test(s), what mixture and/or what level of antibodies were detected as a result of tests (e.g. IgG, IgM, or IgG and IgM), whether the tests were self-reported or obtained from a health facility, the results of the PCR virus test, how old the virus test results are, and the like. The score generated by the application may be obtained from a formula predefined by an administrator and may be static or dynamically update in real time based on the latest collected test results data and latest COVID rules in effect, and other factors. The formula to calculate the COVID risk score may have one or more parameters that are variably set by a venue operator.

For example, the COVID risk score may be based on an equation that incorporates the various components (or factors) of the test results, including results of IgG, IgM, Covid PCR, and when the tests were performed. Antibodies IgG and IgM time since test (and if multiple tests, what the trending is for antibody numbers), and COVID virus (PCR) Fresh/Stale results, i.e., timing of the testing and how recent (freshness or staleness) is the test result (e.g., if within 72 hrs=OK or fresh, if more than 72 hrs, results may become more “stale” on a sliding scale with time). Values may be assigned to each of these components to be used in the Score equation, based on the results. Also, each component may have a weighting factors (W's) associated with its importance such as the following Equation 1 (Eq. 1).

[IgG(W1)+IgM(W2)+PCR(W3)+Fresh/Stale(W4)+Ratio IgG/IgM(W5)]=Score (Eq. 1)

Where the score is normalized to be 0 to 100 (0=lowest risk, 100=highest risk; or 0=highest risk, 100=lowest risk). Other equations and other factors or components and other weighting factors may be used if desired, such as Antibody trends for IgG and IgM, time since antibody test, and any other components that would be helpful in determining a Covid risk score.

In some embodiments, the devices of some users may be configured to only allow sharing of information with other users and not receive information the other users, while the devices of other users may be configured to only allow receiving information and not configured to allow sharing. In some embodiments, the devices may be configured to allow both sharing and receiving of records or information. In some embodiments, the sharing of the file may be temporary and when the person associated with the shared file is a predetermined distance away from the receiving device of the shared file (i.e., further than the broadcast zone), then the file will be deleted, removed, and/or otherwise inaccessible to the receiving device.

In some embodiments, the Passport App may have a timer that determines the length of time the records will be broadcast, e.g., 30 seconds, 1 min., 5 min., 10 min., or the like. When the time expires, the broadcasting of the test results (or other medical records) stops. An alert may be provided to the user, e.g., by text, email, phone call, alarm, or the like indicating that the broadcast has stopped (or both started and stopped), which allows the user to restart the timer or let the broadcast feature remain off. The value of the timer may be set by the user and/or may have a default time, and may be a single timer value for all broadcasts, or may have different timer values based on the type of record being shared.

Advantageously, methods and systems according to the present disclosure will be useful for venue operators for screening persons from entering or accessing or exiting the venue. For example, the security of an office building with a device receive shared information of persons running a proximity-based file sharing application on their mobile device and automatically receive records of the approaching user. Methods and systems of the present disclosure would allow for the office building security to allow individuals that have tested negative for an infectious disease or have tested with the appropriate level or mixture of antibodies entry to the office building (or venue). The proximity-based sharing techniques of the present disclosure allow for limited private sharing of files, such as medical records, so that users with a need to know information about persons in close proximity to the user can easily and automatically be shared in an effective and quick manner.

FIG. 9A illustrates various components (or devices or logic) of a proximity-based file sharing system 900 of the present disclosure, which includes Passport Processing Logic 902, which receives inputs from a plurality of data sources, including but not limited to location tracking sources 904, such as GPS tracking data (or any other location tracking sources that can track the location of the user device and any other mobile devices in a predetermined geographical area having an acceptable distance accuracy), medical records sources 906, such as health care or testing facilities digital portals or servers or the like that can provide digital medical files or records electronically, and virus/disease rules sources 908, such as the CDC (Center for Disease Control) digital portals or servers or the like that can provide digital information about the latest rules and guidelines associated with a relevant virus or other medical health condition or disease.

The Passport Processing Logic (PPL) 902 communicates with Central/Records Server 910 and a User Attributes Server 912 to store and retrieve data used by the PPL. These servers may communicate with a user device 914, such as a user's smart phone to provide data to or receive data from software applications running on the smart phone. The user device may have a Passport software application (Passport App) 916 discussed herein, loaded thereon and a display 918 and communicates with the user. Also, the Passport Processing Logic 902 may communicate with the user device 914 (e.g., for providing alerts or verification codes, or other information), as discussed herein.

The user device 914 may be a computer-based device, which may interact with the user. The user device 914 may be a smartphone, a tablet, a smart TV, a laptop, cable set-top box, or the like. The device 914 may also include the Passport App 916 loaded thereon, for providing a desired graphic user interface or GUI or visualization (as described herein) for display on the user device 914. The Passport App 916 runs on, and interacts with, a local operating system (not shown) running on the computer (or processor) within the user device 914, and may also receive inputs from the user, and may provide audio and video content to audio speakers/headphones (not shown) and the visual display of the user device. The user may interact with the user device 914 using the display 918 (or other input devices/accessories such as a keyboard, mouse, or the like) and may provide input data to the device 914 to control the operation of the Passport App 916 software application running on the user device 914 (as discussed herein).

The display 918 on the user device 914 also interacts with the local operating system on the device 914 and any hardware or software applications, video and audio drivers, interfaces, and the like, needed to view or listen to the desired digital records or information and display the appropriate graphic user interface (GUI) for the Passport App 916 on the user device 914.

The Passport Processing Logic (PPL) 902 executes the logic discussed herein, such as the Sign-up (or registration or log-in) validation logic 100, discussed with FIG. 1, which validates the user and assigns a unique session-based Person ID, the file capture logic 200A, 200B, 200C, discussed with FIGS. 2A and 2B, which obtains the digital records from a third party authorized facility or allows for self-reporting, and the broadcast logic, discussed with FIGS. 3, 4, and 5, which creates the broadcast zone or area and creates the 15 m×15 m detection and file distribution/reception region with other uses on the Passport App 916.

The PPL 902 may save the medical files/records on the Central/Records Server for use by the Passport App on the User Device, as well as may use the data stored on the Central/Records Server 910 to execute the necessary PPL 902 to provide the functions described herein. The PPL 902 may also retrieve user attributes about the user which are stored by the Passport App 916 on the User Attributes Server 912 to execute the necessary PPL 902 to provide the functions described herein.

The PPL 902 may also provide verification codes, data request, and alerts to the user device 914 when needed based on user settings and the necessary PPL 902 described herein. The Passport App 916 running on the user device 914 provides a graphic user interface (GUI) on the display 918, as shown herein with various screen shots, based on the information in the Central/Records Server 910 and User Attributes servers 912 or other servers as needed, and based on inputs and options settings from the user, as described herein.

Also, the system 900 described herein may analyze the data of the user and other users within the broadcast area, continuously in real-time, and provide the user with real-time status updates for the user and for other users in the broadcast area. The user may select various types or forms of information to be displayed about the user and other users as desired and as may be set in the user attributes or settings or preferences or profile for the Passport App 916 for that user, which may be stored on the User Attributes Server 912.

Referring to FIG. 9B, the present disclosure may be implemented in a network environment. In particular, various components of an embodiment of the system 900 of the present disclosure include a plurality of computer-based user devices 914 (e.g., Device 1 to Device N), which may interact with respective users (User 1 to User N). A given user may be associated with one or more of the devices 914. In some embodiments, the Passport App 916 may reside on the device 914 or reside on a remote server and communicate with the user device(s) 914 via the network. In particular, one or more of the user devices 914, may be connected to or communicate with each other through a communications network 920, such as a local area network (LAN), wide area network (WAN), virtual private network (VPN), peer-to-peer network, or the internet, wired or wireless, as indicated by lines, by sending and receiving digital data over the communications network 920. If the user devices 924 are connected via a local or private or secured network, the devices 924 may have a separate network connection to the internet for use by web browsers running on the devices 914. The devices 914 may also each have a web browser to connect to or communicate with the internet to obtain desired content in a standard client-server based configuration to obtain the Passport App 916 or other needed files to execute the logic of the present disclosure. The user devices 914 may also have local digital storage located in the device 914 itself (or connected directly thereto, such as an external USB connected hard drive, thumb drive or the like) for storing data, images, audio/video, documents, and the like, which may be accessed by the Passport App 916 running on the user devices 914.

Also, the computer-based user devices may each communicate via the network with the various Servers and Sources described herein and the Passport Processing Logic 902, and any other network-enabled devices or logics necessary to perform the functions described herein. The servers described herein may be any type of computer server with the necessary software or hardware (including storage capability) for performing the functions described herein. Also, the servers (or the functions performed thereby) may be located, individually or collectively, in a separate server on the network, or may be located, in whole or in part, within one (or more) of the User Devices 914 on the network. In addition, the Location Tracking Sources 904, Medical Records Sources 906, and the Virus/Disease Rules Sources 908, may each communicate via the network 920 with the Passport Processing Logic 902, and with each other or any other network-enabled devices or logics as needed to provide the functions described herein.

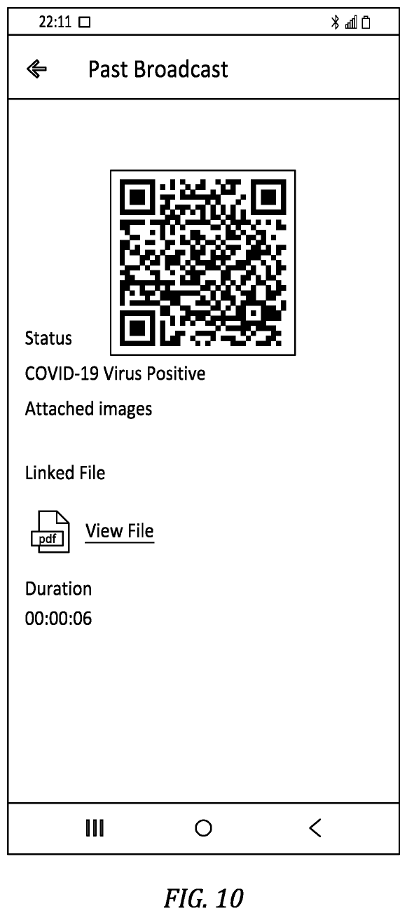

Referring to FIG. 10 shows a sample QR history entry with data and record file, in accordance with embodiments of the present disclosure.

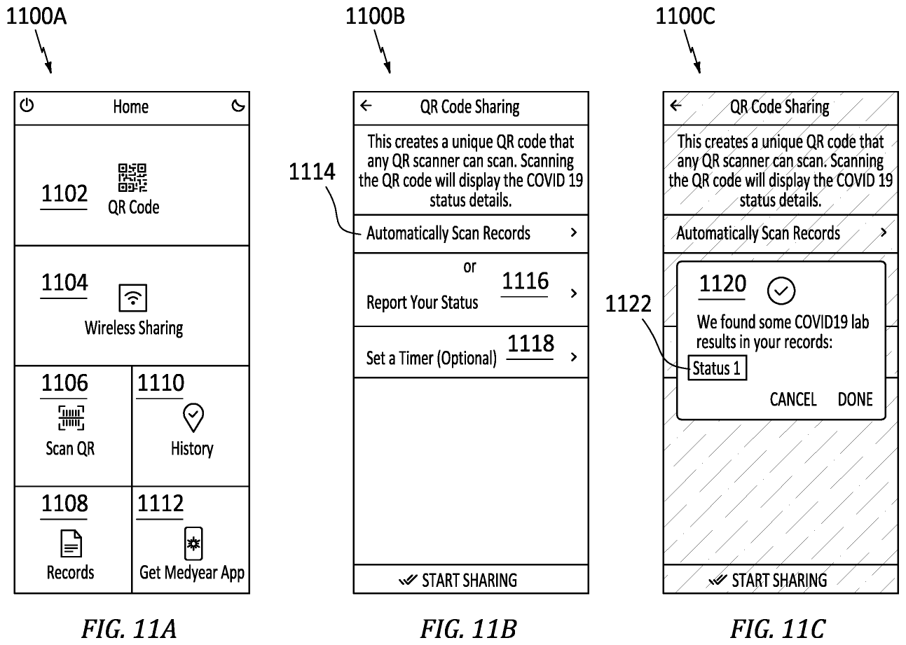

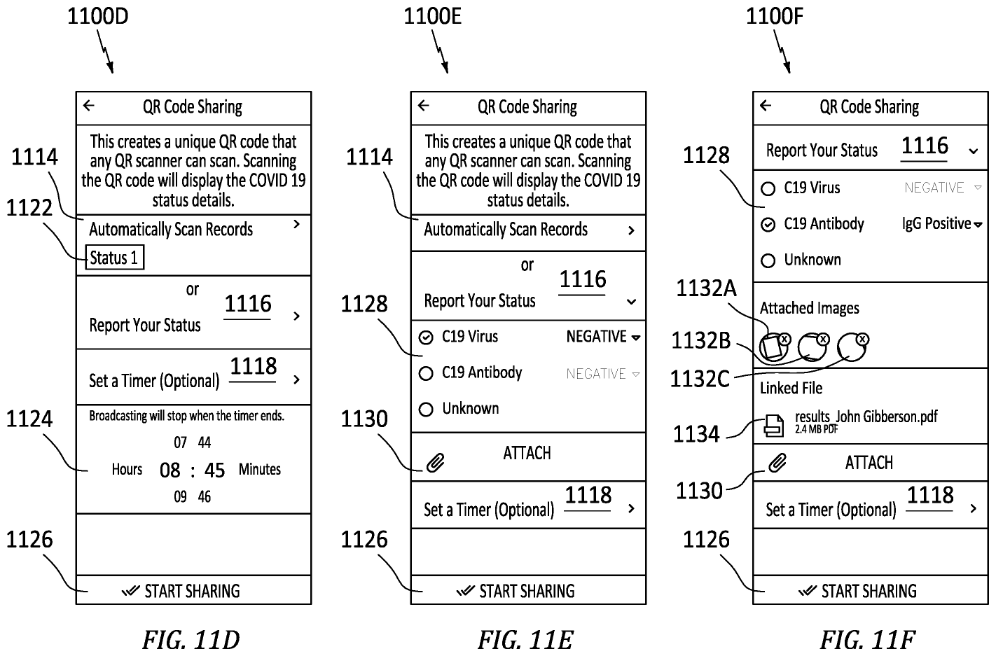

Referring to FIG. 11A, an exemplary screenshot 1100A of a mobile application running on a user device (e.g. 914 of FIGS. 9A and 9B) of a proximity-based file sharing system is shown. In operation, the screenshot 1100A depicts an image (or graphical user interface or “GUI”) displayed by the display (e.g. 918 of FIGS. 9A and 9B) of the user device (914). The screenshot 1100A includes a QR code section 1102, which as discussed above, may be any other machine readable code or graphic, a wireless sharing section 1104, a scan QR section 1106 (or any other machine readable code or graphic), a records section 1108, a history section 1110, and a Get Medyear App section 1112.

A user may select either, or both, the QR code section 1102 and/or the wireless sharing section 1104 to initiate a sharing process, or for configuring the device when sharing. If the user selects the QR code section 1102 or wireless sharing section 1104, the mobile application will cause the user device to display a new image (or GUI), as shown in screenshot 1100B of FIG. 11B (which shows for QR code sharing, but is illustrative of wireless sharing processes as well).

Referring to FIG. 11B, the screenshot shows two sections for user selection for capturing lab or test results in the application: an automatically scan records section 1114 (e.g. 200B of FIG. 2A); and a report your status section 1116 (e.g. 200C of FIG. 2A). The screenshot 1100B also shows a set a timer section 1118 option.

If the user selects the automatically scan records section 1114, the mobile application will cause the user device to display a new image (or GUI), as shown in screenshot 1100C of FIG. 11C. The screenshot 1100C indicates in a results window 1120 the success or failure of the application attempt(s) to automatically retrieve lab or test results for the user through one of the methods discussed above (e.g. 200B of FIG. 2A). In this embodiment, the application retrieved a result 1122 (i.e. “Status 1”).

The application is configured to cause the user device to display a new image (or GUI), as shown in screenshot 1100D of FIG. 11D. The user may manually select the set a timer section 1118 (or the application may automatically select the set a timer section 1118 following retrieval of lab or test results) and the user may be prompted to set an amount of time to broadcast test results in a timer setting section 1124. The user may then select a start sharing section 1126 to begin broadcasting the lab or test results. In some embodiments, instead of setting a timer through the set a timer section 1118, the application may be configured to automatically share the results for a predetermined amount of time without the user being required to manually set a time for sharing.

If the user selects the report your status section 1116 (FIG. 11B), the mobile application will cause the user device to display a new image (or GUI), or as shown in screenshot 1100E of FIG. 11E, a drop-down menu 1128 for manually self-reporting lab or test results. The GUI includes an attach section 1130 that, if selected, will allow the user to upload documents and/or images for supporting the manually self-reported lab or test results, as discussed above in connection method 200C (FIG. 2A). Referring to the screenshot 1100F shown in FIG. 11F, in this embodiment, three images 1132A, 1132B, 1132C and one file 1134 are shown as being attached via the attach section 1130 for supporting the manually self-reported test results of: C19 Virus status negative and C19 Antibody IgG Positive in the drop-down menu 1128. The user is able to set a time through the set a timer section 1118 and begin sharing through the start sharing section 1126 as discussed above.

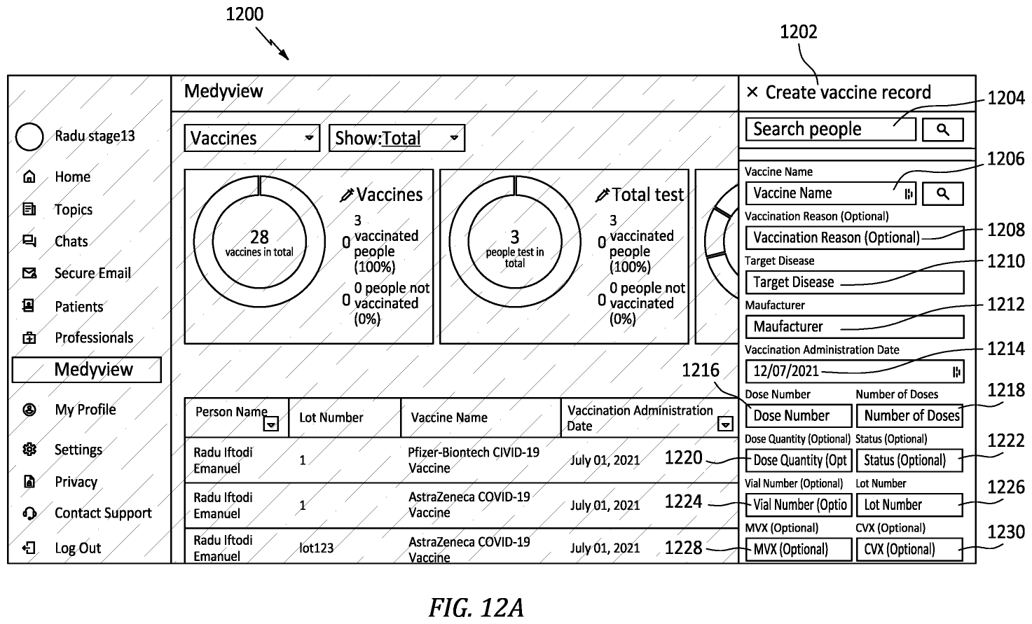

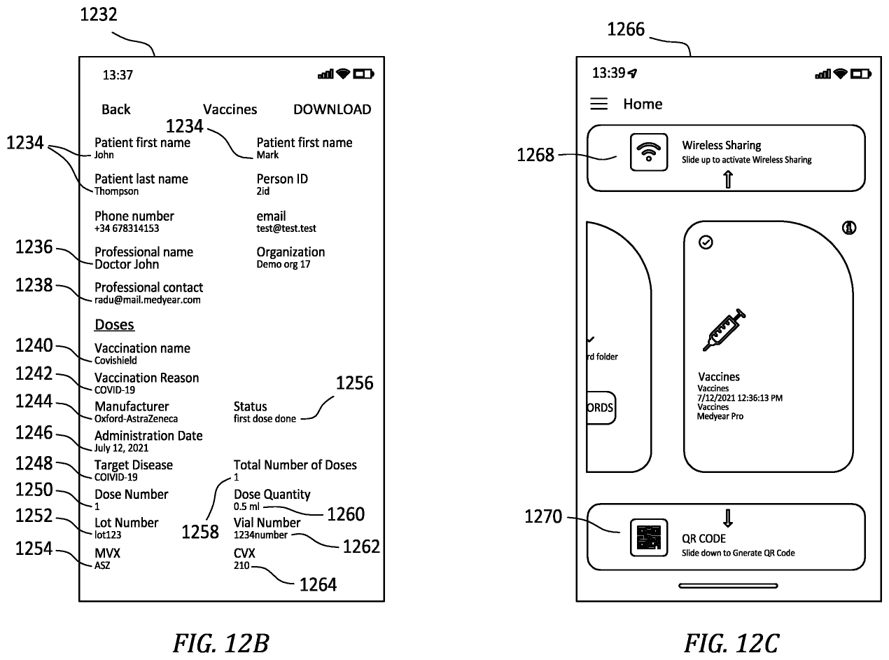

Referring to FIG. 12A, an exemplary screenshot 1200 of an application running on a professional user device for providing medical records (e.g. 906 of FIGS. 9A and 9B) of a proximity-based file sharing system is shown. In operation, the screenshot 1200 depicts an image (or graphical user interface or “GUI”) displayed by the display of the professional user device. The screenshot 1200 includes a create vaccine record section 1202 (but is equally applicable for providing a lab or test result record), which provides a plurality of fields for creating a vaccine record for a patient user (or end user). The fields may include any relevant data for creating a vaccine record (or lab or test result record). In the screen shot 1200, the fields include: a patient name field 1204; a vaccine name field 1206; a vaccination reason field 1208; a target disease field 1210; a manufacturer field 1212; a vaccination administration date field 1214; a dose number field 1216; a number of doses field 1218; a dose quantity field 1220; a status field 1222; a vial number field 1224; a lot number field 1226; an MVX code field 1228 (described hereinafter); and a CVX code field 1230 (described hereinafter). One or more of the fields may be optional and not required to be filled or listed for creating a vaccine record. Other fields and codes, such as other codes provided by the CDC (www.cdc.gov) or other health organizations, may be used if desired provided they provide equivalent function and performance to that described herein.

The fields of the vaccine record section 1202 are appropriately populated by a medical professional or technician during or after a lab or test or administration of a vaccine. The resulting record may be provided as the medical record from the medical record sources 906 (of FIGS. 9A and 9B) for retrieval during an automatic records scanning process as discussed above.

Referring to FIG. 12B, an exemplary screenshot 1232 of a mobile application running on a user device (e.g. 914 of FIGS. 9A and 9B) of a proximity-based file sharing system is shown. In operation, the screenshot 1232 depicts an image (or graphical user interface or “GUI”) displayed by the display (e.g. 918 of FIGS. 9A and 9B) of the user device (914). The screenshot 1232 shows vaccine details of a vaccine record generated by a medical professional or technician that are retrieved during an automatic medical record retrieval process discussed above. In particular, the screenshot 1232 shows: patient name details 1234 corresponding to data entered in the patient name field 1204 (FIG. 12A); professional name details 1236 corresponding to the professional (or professional's office or employer) that administered the vaccine (or lab or test); professional contact details 1238 corresponding to contact information for the professional; vaccination name details 1240 corresponding to data entered in the vaccine name field 1206 (FIG. 12A); vaccination reason details 1242 corresponding to data entered in the vaccination reason field 1208 (FIG. 12A); manufacturer details 1244 corresponding to data entered in the manufacturer field 1212 (FIG. 12A); administration date details 1246 corresponding to data entered in the vaccination administration date field 1214 (FIG. 12A); target disease details 1248 corresponding to data entered in the target disease field 1210 (FIG. 12A); dose number details 1250 corresponding to data entered in the dose number field 1216 (FIG. 12A); lot number details 1252 corresponding to data entered in the lot number field 1226 (FIG. 12A); MVX code details 1254 corresponding to data entered in the MVX code field 1228 (FIG. 12A), e.g., a known alphabetic string that identifies the manufacturer of the vaccine, such as, MVX=SKB for GlaxoSmithKline, MVX=MSD for Merck, and MVX=PFR for Pfizer (more information on MVX codes available at: https://www2a.cdc.gov/nip/IIS/IISStandards/vaccines.asp?rpt=mvx); status details 1256 corresponding to data entered in the status field 1222 (FIG. 12A); total number of doses details 1258 corresponding to data entered in the number of doses field 1218; dose quantity details 1260 corresponding to data entered in the dose quantity field 1220 (FIG. 12A); vial number details 1262 corresponding to data entered in the vial number field 1224 (FIG. 12A); and CVX code details 1264 corresponding to data entered in the CVX code field 1230 (FIG. 12A), e.g., a known numeric string, which identifies the type of vaccine product used (more information on CVX codes is available at: https://www2a.cdc.gov/vaccines/IIS/IISStandards/vaccines.asp?rpt=cvx). Other or additional vaccine code fields may be provided if desired.

Referring to FIG. 12C, an exemplary screenshot 1266 of a mobile application running on a user device (e.g. 914 of FIGS. 9A and 9B) of a proximity-based file sharing system is shown. In operation, the screenshot 1266 shows that the vaccine record containing the vaccine details of FIG. 12B is able to be selected for wirelessly sharing by selecting the wireless sharing section 1268 or for QR code sharing by selecting the QR code section 1270 as discussed above.

In some embodiments, a professional user (or “Medyear Pro” user) is an entity, such as a healthcare provider, that provides medical records (e.g. lab or test results or vaccine records) of the patient(s) to the central records server. A user of a mobile application for sharing digital medical records provided by a professional user may be referred to as a “patient user” (or end user).

Portions of the present disclosure shown herein as being implemented outside the user device, may be implemented within the user device by adding software or logic to the user devices, such as adding logic to the Passport App software or installing a new/additional application software, firmware or hardware to perform some of the functions described herein, such as some or all of the Passport Processing Logic, or other functions, logics, or processes described herein. Similarly, some or all of the Passport Processing Logic of the present disclosure may be implemented by software in one or more of the Central/Records Server or the User Attributes Server, to perform the functions described herein, such as some or all of the Passport Processing Logic, or some or all of the functions performed by the Passport App software in the User Device.

The system, computers, servers, devices, logic and the like described herein have the necessary electronics, computer processing power, interfaces, memory, hardware, software, firmware, logic/state machines, databases, microprocessors, communication links (wired or wireless), displays or other visual or audio user interfaces, printing devices, and any other input/output interfaces, to provide the functions or achieve the results described herein. Except as otherwise explicitly or implicitly indicated herein, process or method steps described herein may be implemented within software modules (or computer programs) executed on one or more general-purpose computers. Specially designed hardware may alternatively be used to perform certain operations. Accordingly, any of the methods described herein may be performed by hardware, software, or any combination of these approaches. In addition, a computer-readable storage medium may store thereon instructions that when executed by a machine (such as a computer) result in performance according to any of the embodiments described herein.

In addition, computers or computer-based devices described herein may include any number of computing devices capable of performing the functions described herein, including but not limited to: tablets, laptop computers, desktop computers, smartphones, mobile communication devices, smart TVs, set-top boxes, e-readers/players, and the like.

Although the disclosure has been described herein using exemplary techniques, algorithms, or processes for implementing the present disclosure, it should be understood by those skilled in the art that other techniques, algorithms and processes or other combinations and sequences of the techniques, algorithms and processes described herein may be used or performed that achieve the same function(s) and result(s) described herein and which are included within the scope of the present disclosure.

Any process descriptions, steps, or blocks in process or logic flow diagrams provided herein indicate one potential implementation, do not imply a fixed order, and alternate implementations are included within the scope of the preferred embodiments of the systems and methods described herein in which functions or steps may be deleted or performed out of order from that shown or discussed, including substantially concurrently or in reverse order, depending on the functionality involved, as would be understood by those reasonably skilled in the art.

It should be understood that, unless otherwise explicitly or implicitly indicated herein, any of the features, characteristics, alternatives or modifications described regarding a particular embodiment herein may also be applied, used, or incorporated with any other embodiment described herein. Also, the drawings herein may not be drawn to scale, unless indicated otherwise.

Conditional language, such as, among others, “can,” “could,” “might,” or “may,” unless specifically stated otherwise, or otherwise understood within the context as used, is generally intended to convey that certain embodiments could include, but do not require, certain features, elements, or steps.

Although the invention has been described and illustrated with respect to exemplary embodiments thereof, the foregoing and various other additions and omissions may be made therein and thereto without departing from the spirit and scope of the present disclosure.

1. A computer-based method for sharing a digital file based on proximity, comprising:
periodically transmitting, by a first mobile device, a geographic location of the first mobile device, wherein the first mobile device is associated with a first user;
periodically transmitting, by a second mobile device, a geographic location of the second mobile device, wherein the second mobile device is associated with a second user;
determining, when the second mobile device is within the predetermined zone of the first mobile device based on the transmitted geographic locations of the first mobile device and the second mobile device;
transmitting, when the second mobile device is within the predetermined zone of the first mobile device, a file associated with the first user to the second mobile device for use by the second user.

2. The method according to claim 1, wherein the file is a medical record of the first user.

3. The method according to claim 1, wherein the file is a plurality of medical records of the first user.

4. The method according to claim 1, wherein the file is a medical record and includes information about a test result of whether the first user tested positive from a test for COVID-19 virus or information about a vaccine record.

5. The method according to claim 4, wherein the file further includes information about when the test for COVID-19 was performed.

6. The method according to claim 4, wherein the file further includes information about whether the first user tested positive for antibody IgG, IgM, or IgG and IgM.

7. The method according to claim 6, wherein the file further includes information about a level of the antibody IgG or IgM.

8. The method of claim 1, further comprising calculating a COVID 19 status of the first user based on test results derived from the file about the first user.

9. The method of claim 1, further comprising calculating a COVID 19 risk score of the first user based on test results derived from the file about the first user.

10. The method of claim 1, further comprising transmitting, when the second mobile device is within the predetermined zone of the first mobile device, a file associated with the second user to the first mobile device for use by the first user.

11. A computer-based method for sharing a digital file based on proximity, comprising:
periodically transmitting, by a first mobile device, a geographic location of the first mobile device to a central server, wherein the first mobile device is associated with a first user;
periodically transmitting, by a second mobile device, a geographic location of the second mobile device to the central server, wherein the second mobile device is associated with a second user;
determining, by the central server, when the second mobile device is within the predetermined zone of the first mobile device based on the transmitted geographic locations of the first mobile device and the second mobile device;
transmitting, by the central server, when the second mobile device is within the predetermined zone of the first mobile device, a file associated with the first user to the second mobile device for use by the second user.

12. The method according to claim 11, wherein the file is a medical record of the first user.

13. The method according to claim 11, wherein the file is a plurality of medical records of the first user.

14. The method according to claim 11, wherein the file is a medical record and includes information about a test result of whether the first user tested positive from a test for COVID-19 virus or information about a vaccine record.

15. The method according to claim 14, wherein the file further includes information about when the test for COVID-19 was performed.

16. The method according to claim 14, wherein the file further includes information about whether the first user tested positive for antibody IgG, IgM, or IgG and IgM.

17. A computer-based method for sharing a digital file based on proximity, comprising:
retrieving, by a first mobile device, a digital file comprising lab or test results or vaccine record of a first user;
generating, by the first mobile device, a machine readable code or graphic;
scanning, by a second mobile device, the machine readable code or graphic;
retrieving, by the second mobile device, the lab or test results or vaccine record of the first user based on the machine readable code or graphic.

18. The method according to claim 17, wherein the machine readable code or graphic is displayed on a display of the first mobile device during the generating the machine readable code or graphic.

19. The method of claim 18, wherein the machine readable code or graphic is a QR code.

20. The method of claim 17, wherein the second mobile device retrieves the lab or test results of the first user from a central server.

21. The method according to claim 1 wherein the predetermined zone comprises a predetermined number of Level-9 geohash cells, each cell defining a predetermined distance or area.

22. (canceled)

23. The method according to claim 1 wherein the predetermined zone comprises a predetermined broadcast zone comprising an area that is approximately 14.34 meters wide by approximately 14.34 meters long directly surrounding the geographic location of the first mobile device.
